(19) United States
(12) Patent Application Publication
(10) Pub. No.: US 2026/0297692 A1
(43) Pub. Date: Oct. 1, 2026

(54) PROBE SET FOR NUCLEIC ACID TESTING OF SARS-COV-2 CORONAVIRUSES AND USES

(71) Applicant: FuZhou University, FUJIAN (CN)

(72) Inventors: Jun LIN, Fujian (CN); Tao WANG, Fujian (CN); Wenqian JIANG, Fujian (CN)

(73) Assignee: FuZhou University, FUJIAN (CN)

(21) Appl. No.: 19/577,363

(22) Filed: Mar. 25, 2026

(30) Foreign Application Priority Data
Mar. 27, 2025 (CN) 202510374890.0

(51) Int. Cl. C12Q 1/70

(52) U.S. Cl. CPC C12Q 1/701; C12Q 2600/156

(57) ABSTRACT

The present invention discloses a probe set for nucleic acid testing of SARS-CoV-2 coronaviruses and uses. A crRNA sequence for nucleic acid testing of SARS-CoV-2 coronaviruses is any one of the following: LbucrRNA1, LbucrRNA2, LbucrRNA3 and LbucrRNA4 for an S gene, as well as N1crRNA and N2crRNA for an N gene of a mutant strain EG.5.1.1, with sequences shown in the sequence listing. Based on the ability of the LbuCas13a protein to cleave free RNAs and target specific RNA sequences, the present invention establishes an Cas13a-based immunochromatographic method for nucleic acid testing using a visual testing method based on colloidal carbon immunochromatographic test strips. The method enables rapid testing of SARS-CoV-2 coronaviruses within 90 min, providing a novel, easy and rapid testing method for disease prevention and control. Moreover, the method is simple to operate and does not require the assistance of large instruments and equipment.

### CROSS-REFERENCES TO RELATED APPLICATIONS

This application claims the priority benefit of China application serial no. 202510374890.0, filed on Mar. 27, 2025. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Feb. 26, 2026, is named 164851-US-Sequence_Listing and is 6,366 bytes in size.

### BACKGROUND

### Technical Field

The present invention belongs to the field of molecular biological testing, and in particular, relates to a probe set for nucleic acid testing of SARS-CoV-2 coronaviruses and uses.

### Description of Related Art

As a novel respiratory RNA virus, a SARS-CoV-2 coronavirus has high transmissibility and mutagenicity. During disease prevention and control periods, effective and accurate testing of the viruses is an effective means. Reverse transcription-quantitative polymerase chain reaction (RT-qPCR), a gold standard for testing the SARS-CoV-2 coronavirus, is an accurate and reliable method, which however usually takes a long time, requires complex operations and relies on instruments and equipment. Hence, it is of great significance to develop a low-cost, convenient and efficient method for testing of SARS-CoV-2 coronavirus variants, to control respiratory diseases caused by this virus.

In terms of specificity and operability, clustered regularly interspaced short palindromic repeats (CRISPR)-based techniques have proven to show unique advantages in molecular diagnostics as compared to traditional PCR methods. Due to the high specificity of Cas/guide-RNA in target recognition, CRISPR-targeted testing methods are used to identify the point mutations in the SARS-CoV-2 coronavirus. As a key effector in a CRISPR system, a Cas13a protein has a unique ribonuclease activity and can precisely cleave a target RNA under the guidance of a CRISPR RNA (crRNA), and at the same time, the non-specific cleavage activity of the Cas13a protein can be stimulated to cleave an RNA on the surface of the Cas13a protein. Among homologous Cas13a proteins, a LbuCas13a protein shows an excellent cleavage performance, and it allows for sensitive and specific cleavage of nucleic acids. At the same time, the LbuCas13a protein can be combined with an immunochromatographic test strip to achieve conversion and amplification of cleavage signals and rapid and significant presentation of testing results, showing great potential in the rapid and low-cost testing of the SARS-CoV-2 coronaviruses.

Methods for interpreting the immunochromatographic test strip for Cas13a-based testing of RNA viruses in China and abroad mainly include a “visible-line method”, i.e., observing a band on a test line (T line for short) to interpret a positive nucleic acid testing result. This method facilitates intuitive interpretation of the testing result. That is, when a target RAN is present, the Cas13a protein cleaves the RNA; when a probe is not completely cleaved, the intact probe is retained at a control line (C line for short), the cleaved free marker-containing probe is captured by specific antibodies at the T line. In this way, bands appear at both the C and T lines, indicating a positive interpretation result. However, when the probe is completely cleaved and broken, no colloidal gold accumulation occurs at the C line, and only a band appears at the T line, without any band appearing at the C line. This does not conform to the rigor of in vitro diagnosis.

In order to conform to the rigor of in vitro diagnosis, if the testing mode of “line-free method” is used (that is, no band appears at the “T” line, leading to the interpretation of a positive nucleic acid testing result), an opposite way of thinking should be adopted when the test strip is laid out to test the specific antibody corresponding to the probe. When a target RNA does not exist, Cas13a cannot perform cleavage, the probe remains intact and is captured at the T line of the test strip, and excess colloidal gold-labeled antibodies are captured at the C line, such that bands appear at both the C and T lines, leading to the interpretation of a positive result. When a target RNA exists, Cas13a performs cleavage, the probe is completely cleaved, and the labeled end of the probe is not captured by the antibody at the T line, but migrates freely to the C line and is captured by the antibody at the C line, such that the band appears at the C line rather than the T line, resulting in the interpretation of a positive result.

### SUMMARY

An object of the present invention is to provide a probe set for nucleic acid testing of SARS-CoV-2 coronaviruses and uses. Based on the ability of an LbuCas13a protein to cleave free RNAs and targeting specific RNA sequences, the present invention establishes an immunochromatographic method for testing of a Cas13a nucleic acid by using a colloidal carbon immunochromatographic test strip as a visual testing method.

To achieve the above object, the technical solutions of the present invention are as follows.

The present invention first provides a probe set for nucleic acid testing of SARS-CoV-2 coronaviruses, wherein a crRNA sequence for nucleic acid testing of SARS-CoV-2 coronaviruses is any one of the following: LbucrRNA1, LbucrRNA2, LbucrRNA3 and LbucrRNA4 for an S gene, as well as N1crRNA and N2crRNA for an N gene of a mutant strain EG.5.1.1, with sequences as follows:

| Name | Sequence (5′-3′) |
|---|---|
| LbucrRNA1 | GACCACCCCAAAAATGAAGGGGACTAAAACGAATT |
| | CCAAGCTATAACGCAGCCTGTAA |
| | (SEQ ID NO: 1) |
| LbucrRNA2 | GACCACCCCAAAAATGAAGGGGACTAAAACCTTGC |
| | TGTGGAAGAAAGTGAGTCTTGA |
| | (SEQ ID NO: 2) |
| LbucrRNA3 | GACCACCCCAAAAATGAAGGGGACTAAAACGCGAT |
| | TTGTCTGACTTCATCACCTCTA |
| | (SEQ ID NO: 3) |
| LbucrRNA4 | GACCACCCCAAAAATGAAGGGGACTAAAACCCTAT |
| | CAATTTGCACTTCAGCCTCAAC |
| | (SEQ ID NO: 4) |
| N1crRNA | GACCACCCCAAAAATGAAGGGGACTAAAACTGTCC |
| | TTTTTAGGCTCTGTTGGTGGGA |
| | (SEQ ID NO: 5) |
| N2crRNA | GACCACCCCAAAAATGAAGGGGACTAAAACTGAAC |
| | TGTTGCGACTACGTGATGAGGA |
| | (SEQ ID NO: 6) |

The present invention further provides a kit for nucleic acid testing of SARS-CoV-2 coronaviruses, including the probe set for nucleic acid testing of SARS-CoV-2 coronaviruses as described in the present invention.

Further, the kit for nucleic acid testing of SARS-CoV-2 coronaviruses includes a line-free colloidal carbon immunochromatographic test strip and a CRISPR reaction system, wherein the CRISPR reaction system includes the probe set for nucleic acid testing of SARS-CoV-2 coronaviruses as described in the present invention; the line-free colloidal carbon immunochromatographic test strip sequentially includes a sample pad, a conjugate pad including a colloidal carbon-labeled rabbit anti-biotin antibody, an NC membrane including T and C lines, and absorbent paper in a sample flowing direction;
when a probe is FAB-polyU-11nt, the T line is formed by a rabbit anti-6-FAM polyclonal antibody, and the C line is formed by a goat anti-rabbit IgG;
when a probe is DB-polyU-11nt, the T line is formed by an anti-digoxigenin antibody, and the C line is formed by a goat anti-rabbit IgG;
when a probe is TB-polyU-11nt, the T line is formed by an anti-TAMRA antibody [5G5], and the C line is formed by a goat anti-rabbit IgG;
when a probe is CB-polyU-11nt, the T line is formed by an anti-Cy5 antibody [CY5-15], and the C line is formed by a goat anti-rabbit IgG; and
each probe has a sequence as follows:

| Name | Sequence (5′-3′) |
|---|---|
| FAB-polyU-11 nt | FAM-UUUUUUUUUUU-Biotin |
| TB-polyU-11 nt | TAMRA-UUUUUUUUUUU-Biotin |
| DB-polyU-11 nt | Dig-UUUUUUUUUUU-Biotin |
| CB-polyU-11 nt | Cy5-UUUUUUUUUUU-Biotin. |

Further, the CRISPR reaction system is as follows,

First system for S gene

| Component | Volume |
|---|---|
| 10 × Cas13a buffer | 2 μL |
| Cas13a protein (3 mg/μL) | 2 μL |
| LbucrRNA1/LbucrRNA2/LbucrRNA3/ | 4 μL |
| LbucrRNA4 Mixture (500 ng/μL) | |
| ddH₂O | 12 μL |

First system for N gene

| Component | Volume |
|---|---|
| 10 × Cas13a buffer | 2 μL |
| Cas13a protein (3 mg/μL) | 2 μL |
| N1crRNA/N2RNA Mixture (500 ng/μL) | 2 μL |
| ddH₂O | 14 μL |

after being prepared, the first system is added to a clean RNase-free PCR tube, and then placed in a PCR instrument to react at 37° C. for 10 min to obtain a step 1 system, and after completion of reaction, a Cas13a/crRNA mixture is obtained and added to a second system for step 2,

Second system for S or N gene

| Component | Volume | |
|---|---|---|
| Target RNA | 5 | μL |
| 10 × Cas13a buffer | 8 | μL |
| 40 nM RNA reporter | 10 | μL |
| Cas13a/crRNA mixture (from step 1) | 20 | μL |
| RNase Inhibitor (40 U/μL) | 2.5 | μL |
| ddH₂O | 54.5 | μL |

after being prepared, the second system is placed in the PCR instrument to react at 37° C. for 90 min and then at 4° C. for 2 min to terminate the reaction, the prepared colloidal carbon immunochromatographic test strip is inserted, and test results are read within 5-10 min.

The present invention further provides uses of the kit in nucleic acid testing of SARS-CoV-2 coronaviruses, and the uses are not intended for the diagnosis or treatment of diseases.

According to the above technical solutions used in the present invention, an LbuCas13a-based immunochromatographic testing method is constructed by taking the LbuCas13a protein as an effector protein for testing, combining the CRISPR-LbuCas13a Cas13a-based testing with the interpretation of the immunochromatographic test strip, and presenting the testing result by a “line-free method” (i.e., interpreting a positive result in case of no band appearing at the T line of the test strip).

### Beneficial Effects of the Present Invention

1. The colloidal carbon immunochromatographic test strip is successfully constructed and is combined with the Cas13a reaction to establish an LbuCas13a-based immunochromatographic testing method; and five inactivated SARS-CoV-2 coronavirus-positive samples and 4 negative samples are screened by the fluorescent PCR method and colloidal gold test strips are tested; and the results indicate that both positive and negative samples are successfully detected with 100% consistency compared to the fluorescent PCR method and colloidal gold test strips, demonstrating the accuracy of this method.
2. The qPCR quantification and digital PCR results are analyzed to determine that the minimum detection limit of the LbuCas13a-based immunochromatographic testing method is 381.75 copies/μL.
3. The testing mode of “line-free method” combining Cas13a in the present invention does not require amplification of the target, effectively preventing aerosol contamination during amplification.
4. The sensitivity and specificity tests of the SARS-CoV-2 coronavirus variants using the method of the present invention demonstrate that the method can detect the SARS-CoV-2 coronavirus variants rapidly, sensitively and accurately.

### BRIEF DESCRIPTION OF THE DRAWINGS

FIG. 1 shows electrophoretograms of PCR products of in vitro transcription templates of crRNA, where in A, M indicates a Takara 20 bp DNA ladder marker, and lanes 1 to 4 represent a LwcrRNA1 in vitro transcription template, a LwcrRNA2 in vitro transcription template, a LwcrRNA3 in vitro transcription template, and a LwcrRNA4 in vitro transcription template, respectively; and in B, M indicates a Takara 20 bp DNA marker, lanes 1 to 3 represent a LbucrRNA1 in vitro transcription template, a LbucrRNA2 in vitro transcription template, and a LbucrRNA3 in vitro transcription template, respectively, lanes 4 and 5 represent forward and reverse primer fragments without PCR, respectively, and lane 6 represents a mixture of forward and reverse primer fragments without PCR.

FIG. 2 shows a graph of Cas13a protein activity testing, with A: commercial LwacrRNA protein activity testing; and B: LbuCas13a protein activity testing of the present invention.

FIG. 3 shows LbuCas13a protein activity testing, with 1: a negative control group; and 2: a target RNA-containing experimental group.

FIG. 4 shows a schematic diagram of the testing principle of a singleplex CRISPR test strip.

FIG. 5 shows the specificity assay of LbuCas13a by immunochromatography, with 0: water+probe; 1: water+probe+lbuCas13a protein; 2: water+probe+lbuCas13a protein+crRNA; 3: water+probe+lbuCas13a protein+crRNA+ssRNA.

FIG. 6 shows a graph of testing results of fluorescent PCR for SARS-CoV-2 coronaviruses and images of testing results of some test strips.

FIG. 7 shows laboratory samples for Cas13a-based immunochromatographic testing, with 0: negative control; 2-6: positive RNA samples of inactivated SARS-CoV-2 coronaviruses determined by fluorescent PCR methods numbered 002 to 006; 1: negative sample numbered 001; 7: negative sample numbered 007; 8: negative sample numbered 008; and 9: negative sample numbered 009.

FIG. 8 shows a graph of qPCR standard curve results, with A: qPCR amplification curves; and B: qPCR standard curve.

FIG. 9 shows a graph of one-dimensional digital PCR results.

FIG. 10 shows an image of sensitivity results of Cas13a-based immunochromatographic testing, with 0: negative control; and 1-7: RNA samples of inactivated SARS-CoV-2 coronaviruses, specifically: 1:6108 copies/μL sample; 2:3054 copies/μL sample; 3:1527 copies/μL sample; 4:763.5 copies/μL sample; 5:381.75 copies/μL sample; 6:190 copies/μL sample; and 7:9595 copies/μL sample.

FIG. 11 shows an image of testing results of inactivated SARS-CoV-2 coronavirus and influenza virus samples, with a T1 line corresponding to a probe TARMA test group (with the SARS-CoV-2 coronavirus as a test sample); a T2 line corresponding to a Dig-group test line (with the influenza virus as a test sample); 0: negative control; and 1-8: SARS-CoV-2 coronavirus and influenza virus samples.

### DESCRIPTION OF THE EMBODIMENTS

The present invention is further illustrated below in conjunction with the accompanying drawings and embodiments. The following experimental methods without clearly indicated specific conditions are in accordance with the conventional experimental conditions in the art or the conditions recommended by the manufacturers.

Goat anti-rabbit IgGs and rabbit anti-6-FAM polyclonal antibodies were purchased from Sangon Bioengineering Co., Ltd.; and anti-Digoxigenin antibodies, anti-TAMRA antibodies [5G5], and anti-Cy5 antibodies [CY5-15] were purchased from the manufacturer abcam.

LwaCas13a proteins were purchased commercial proteins, and LbuCas13a proteins were obtained by self-expression and purification through an Escherichia coli clonal expression system with reference to the following literature.
[1] Liu L, Li X, Ma J, et al. The Molecular Architecture for RNA-Guided RNA Cleavage by Cas13a. [J]. Cell, 2017:714.DOI: 10.1016/j.cell.2017.06.050.
[2] Adler B A, Hessler T, Cress B F, Lahiri A, Mutalik V K, Barrangou R, Banfield J, Doudna J A. Broad-spectrum CRISPR-Cas13a enables efficient phage genome editing. Nat Microbiol. 2022: 7 (12): 1967-1979. doi: 10.1038/s41564-022-01258-x.

### Example 1: Preparation of crRNA and Extraction of Target RNA

(1) To verify the activity of LbuCas13a protein, a corresponding crRNA was designed based on the pseudovirus of SARS-CoV-2 coronavirus (S gene) (obtained by inserting a portion of a phage genome and a SARS-CoV-2 coronavirus (S gene) into a pSE380 plasmid, followed by expression in Escherichia coli Er2566 to obtain pseudovirus particles) provided by the present research group. A method for preparing crRNA includes: first synthesizing the corresponding DNA Oligo of RNA, forming a double-stranded DNA by PCR, and then performing in vitro transcription to prepare the crRNA. DNA template fragments corresponding to the RNA were synthesized by Anhui General Biology Co., Ltd. The sequences of DNA template are shown in Table 1.

TABLE 1
Sequences of synthesized DNA templates

| Name | Sequence (5′-3′) |
|---|---|
| LwcrRNA1 template | GATTTAGACTACCCCAAAAACGAAGGGGACTAAAA |
| | CCTACCGGCCTGATAGATTTCAGTTGAAA |
| LwcrRNA2 template | GATTTAGACTACCCCAAAAACGAAGGGGACTAAAA |
| | CCAACACCATTAGTGGGTTGGAAACCATA |
| LwcrRNA3 template | GATTTAGACTACCCCAAAAACGAAGGGGACTAAAA |
| | CACCTTCAACACCATTACAAGGTGTGCTA |
| LbucrRNA1 template | GACCACCCCAAAAATGAAGGGGACTAAAACGAATT |
| | CCAAGCTATAACGCAGCCTGTAA |
| LbucrRNA2 template | GACCACCCCAAAAATGAAGGGGACTAAAACCTTGC |
| | TGTGGAAGAAAGTGAGTCTTGA |
| LbucrRNA3 template | GACCACCCCAAAAATGAAGGGGACTAAAACGCGAT |
| | TTGTCTGACTTCATCACCTCTA |
| LbucrRNA4 template | GACCACCCCAAAAATGAAGGGGACTAAAACCCTAT |
| | CAATTTGCACTTCAGCCTCAAC |

With the synthesized DNA fragments as templates, PCR amplification (with an upstream primer containing a T3 promoter sequence at a 5′-terminal) was performed on these synthesized DNA fragments to obtain DNA fragments containing the T3 promoter sequences. The primers used for amplification were synthesized by Anhui General Biology Co., Ltd., with primer sequences shown in Table 2.

TABLE 2
Amplification primers for synthesized DNA templates

| Primer | Sequence (5′-3′) |
|---|---|
| LwFor | AATTAACCCTCACTAAAGGGATTTAGACTACCC |
| | CAAAAACGAAG |
| LwcrRNA1-Rev | TTTCAACTGAAATCTATCAGGCC |
| LwcrRNA2-Rev | TATGGTTTCCAACCCACTAATG |
| LwcrRNA3-Rev | TAGCACACCTTGTAATGGTG |
| LbuFor | AATTAACCCTCACTAAAGGGGACCACCCCAAAA |
| | ATGAAGGG |
| LbucrRNA1-Rev | TTACAGGCTGCGTTATAGCTTG |
| LbucrRNA2-Rev | TCAAGACTCACTTTCTTCCACAG |
| LbucrRNA3-Rev | TAGAGGTGATGAAGTCAGACAAA |
| LbucrRNA4-Rev | GTTGAGGCTGAAGTGCAAATTG |

Referring to Tables 3 and 4, a PCR amplification system was formulated for PCR amplification, and amplification products were detected by agarose gel electrophoresis.

TABLE 3
Double-stranded amplification system for
in vitro transcription template crRNA

| Component | Volume | |
|---|---|---|
| Megai Pro Fidelity 2 × PCR Master Mix | 25 | μL |
| DNA template (5 μM) | 1 | μL |
| For (10 μM) | 2 | μL |
| Rev (10 μM) | 2 | μL |
| ddH₂O | 20 | μL |

TABLE 4
Double-stranded amplification procedures
for in vitro transcription template crRNA

| Step | Detailed procedures |
|---|---|
| 1^{st} section | 95° C., 5 min |
| 2^{nd} section (−1° C./cycles) | 95° C., 15 s; 58-48° C., 15 s; |
| | 72° C., 30 s |
| 3^{rd} section (25 cycles) | 95° C., 15 s; 48° C., 15 s; |
| | 72° C., 30 s |
| 4^{th} section | 72° C., 5 min |

(2) The RNA of the pseudovirus of SARS-CoV-2 coronavirus (S gene) was extracted using Beyotime RNAeasy™ viral RNA extraction kit, measured for concentration by Nanodrop 2000, and stored at −80° C.
(3) Purification of double-stranded products of in vitro transcription template DNA
(i) 150 μL of 1×TE (pH 8.0) was thoroughly mixed with the above double-stranded PCR amplification products.
(ii) 5 μL of a nucleic acid precipitation aid was added to a 195 μL system.
(iii) 20 μL of 5 M sodium chloride was added to the mixture system and mixed thoroughly by pipetting.
(iv) 2× volume of absolute ethanol was added to the above mixture system, mixed thoroughly by pipetting and then preserved in a freezer at −80° C. for 40 min.
(v) The mixture was removed from the freezer at −80° C. and centrifuged at 12,000×g for 10 min to collect nucleic acids, and the supernatant was removed using a pipette tip on a clean bench.
(vi) 1 mL of 70% ethanol was added to pellets, and a resulting mixture was gently inverted and then centrifuged at 12,000×g for 1 min. A supernatant was carefully discarded, and a lid was removed for airing for 5 min, to remove residual ethanol.
(vii) The nucleic acid pellet was dissolved in 10 μL of DEPC Treated Water solvent and let stand for 5 min to obtain a purified in vitro transcription template, which was measured for concentration using Nanodrop 2000.

### Example 2: In Vitro Transcription of crRNA

(1) The purified double-stranded in vitro transcribed template crRNA was used to prepare an RNA transcription reaction system. The transcription reaction system is shown in Table 5.

TABLE 5
RNA transcription reaction system

| Component | Volume | |
|---|---|---|
| T3 RNA Polymerase | 4 | μL |
| NTP Mix (25 mM) | 0.8 | μL |
| Reaction buffer (10×) | 4 | μL |

| | |
|---|---|
| Template DNA | About 2 μg |
| ddH₂O | Up to 40 μL |

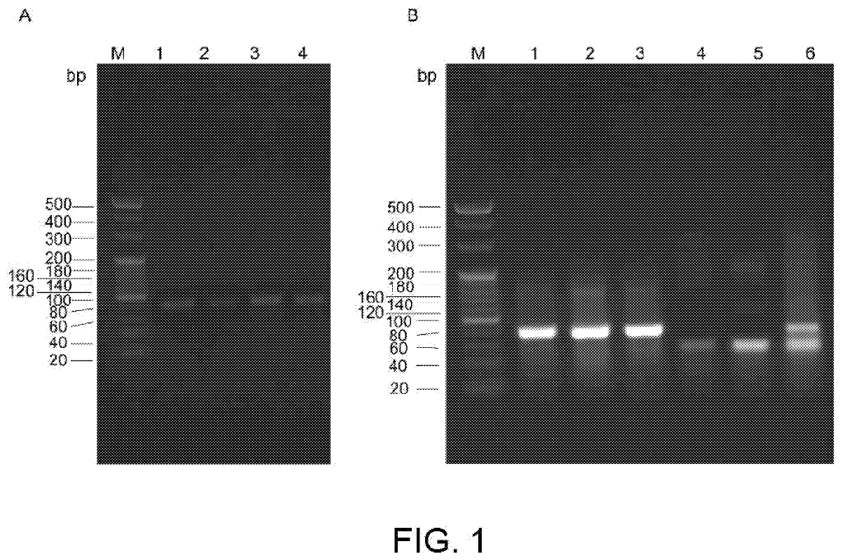

A prepared mixture was placed in an incubator at 37° C. to react for 16 h.
(2) Purification of in vitro transcription products
(i) 4 μL of DNase I (100 mg/mL) was added to the in vitro transcription system, and incubated at 37° C. to react for 15 min, followed by removal of template DNA.
(ii) 135 μL of DEPC Treated Water and 15 μL of a 3 M acetic acid solution (pH 5.2) were added to the reaction system, and thoroughly mixed by pipetting.
(iii) 200 μL of phenol chloroform (25:24:1=phenol:chloroform:isopentanol) was added to the reaction system and mixed thoroughly.
(iv) The reaction mixture was centrifuged at 12,000×g for 10 min, until a clear separation of layers. An upper aqueous layer was transferred to a clean 1.5 mL centrifuge tube.
(v) 2× volume of absolute ethanol was added to the supernatant, a resulting mixture was preserved in a freezer at −80° C. for 40 min and centrifuged at 12,000×g for 20 min, a supernatant was removed, and nucleic acid pellets were collected.
(vi) The nucleic acid pellets were washed with 1 mL of pre-cooled 70% ethanol and centrifuged at 12,000×g for 1 min, and a supernatant was discarded.
(vii) RNA pellets were resuspended in 20 μL of DEPC Treated Water, measured for concentration using Nanodrop 2000, and preserved at −80° C.
(3) Results: For synthesis of crRNA using in vitro transcription, DNA sequences corresponding to crRNA were first amplified by PCR, to obtain double-stranded template products containing T3 promoters, as shown in FIG. 1. A target fragment size was theoretically 78 bp. The results show that compared to fragments untreated by PCR, the size of fragment products of interest matches the theoretical value when referenced against a 20 bp DNA ladder marker, demonstrating the suitability for subsequent in vitro transcription. The in vitro transcribed crRNA was purified by phenol-chloroform extraction, resulting in a concentration of about 500 ng/μL, and the purified crRNA was stored at −80° C. for a subsequent protein activity verification experiment.

### Example 3: Activity Assay of LbuCas13a Protein

(1) To verify the accuracy of the crRNA preparation and reaction system, a commercial LwaCas13a protein, an S-gene RNA and LwcrRNA, together with fluorescent reporter molecules, were used to formulate a Cas13a protein reaction system. The sequences of the fluorescent reporter molecules are shown in Table 6, and the reaction system is shown in Tables 7 and 8.

TABLE 6
Sequences of the fluorescent reporter molecules

| Name | Sequence (5′-3′) |
|---|---|
| FQ-polyU-11 nt | FAM-UUUUUUUUUUU-BHQ1 |
| FB-polyU-11 nt | FITC-UUUUUUUUUUU-Biotin |
| TB-polyU-11 nt | TAMRA-UUUUUUUUUUU-Biotin |

TABLE 7
First reaction system for LwaCas13a protein

| Component | Volume | |
|---|---|---|
| 10 × Cas13a buffer | 2 | μL |
| Cas13a protein (200 ng/μL) | 10 | μL |
| LwcrRNA1/LwcrRNA2/LwcrRNA3 Mixture (500 | 3 | μL |
| ng/μL) | | |
| ddH₂O | 5 | μL |

The above reaction system was placed in a PCR instrument, and a reaction procedure was set as: 37° C., 10 min. This was used as a step 1 system. After completion of the reaction, a reaction system was added following the table below to perform step 2.

TABLE 8
Second reaction system for LwaCas13a protein

| Component | Volume | |
|---|---|---|
| ssRNA | 100 | ng |
| 10 × Cas13a buffer | 8 | μL |
| 10 μM RNA reporter | 2 | μL |
| Cas13a/crRNA mixture (from step 1) | 20 | μL |
| RNase Inhibitor (40 U/μL) | 2.5 | μL |

| | |
|---|---|
| ddH₂O | Up to 100 μL |

For a blank control group, an equal volume of RNase-Free H₂O was used in place of LwcrRNA, and ssRNA and LwaCas13a protein and added to the reaction system. The probe used in the system was FQ-polyU-11nt. Arte all reaction systems were formulated well, these reaction systems were added to a clean black microplate, mixed thoroughly by pipetting, and centrifuged briefly to remove bubbles. A fluorescence microplate reader was used with an excitation wavelength of 492 nm and an emission wavelength of 518 nm, and the reaction procedure was set as follows: 37° C. for 2 h, with fluorescence detection every 5 min.

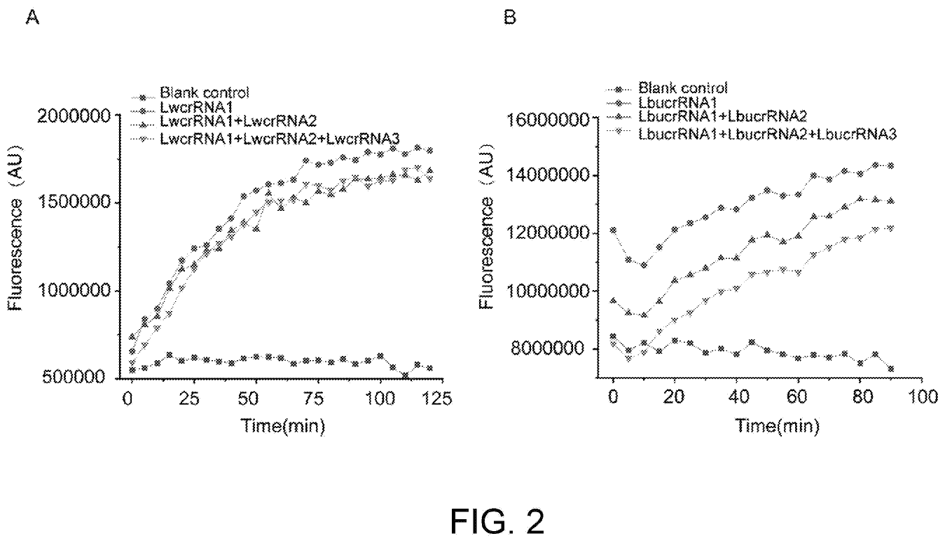

The fluorescence change results are shown in A of FIG. 2. As can be seen, when the target RNA exists, the commercial LwaCas13a protein enables normal probe cleavage as compared to the blank control group, with significantly enhanced fluorescence signals. These results demonstrate the accuracy of the crRNA preparation and testing system, which allows for testing and signal capture of Cas13a protein.
(2) Verification of LbuCas13a protein activity.
(i) The purified LbuCas13a protein, S-gene RNA and LbucrRNA, together with fluorescence reporter molecules, were used to formulate a Cas13a protein reaction system (tested by a microplate reader). The reaction system is the same as those in Tables 7 and 8.
(ii) The purified LbuCas13a protein, S-gene RNA and LbucrRNA, together with fluorescence reporter molecules, were used to formulate a Cas13a protein reaction system (tested by a test strip). The reaction system is the same as those in Tables 9 and 10.

TABLE 9
First reaction system for LbuCas13a protein

| Component | Volume | |
|---|---|---|
| 10 × Cas13a buffer | 2 | μL |
| Cas13a protein (3 mg/μL) | 2 | μL |
| LbucrRNA1/LbucrRNA2/LbucrRNA3/LbucrRNA4 Mixture | 4 | μL |
| (500 ng/μL) | | |
| ddH₂O | 12 | μL |

The above reaction system was placed in a PCR instrument, and a reaction procedure was set as: 37° C., 10 min. This was used as a step 1 system. After completion of the reaction, a reaction system was added following the table below to perform step 2.

TABLE 10
Second reaction system for LbuCas13a protein

| Component | Volume | |
|---|---|---|
| ssRNA | 5 | μL |
| 10 × Cas13a buffer | 8 | μL |
| 100 nM RNA reporter | 10 | μL |
| Cas13a/crRNA mixture (from step 1) | 20 | μL |
| RNase Inhibitor(40 U/μL) | 2.5 | μL |

| | |
|---|---|
| ddH₂O | Up to 100 μL |

For the blank control group, an equal volume of RNase-Free H₂O was used in place of LbucrRNA, ssRNA and LbuCas13a proteins and added to the reaction system. The probe used in the system was FB-polyU-11nt. Arte all reaction systems were formulated well, these reaction systems were added to a clean PCR tube, and placed in a thermostatic incubator to react at 37° C. for 2 h. After completion of the reaction, a dedicated Tiosbio® Cas12/Cas13 nucleic acid test strip was inserted into the PCR tube containing a reaction solution for testing, and a testing result was read within 5-10 min.

The results are shown in B of FIG. 2. The enzyme-linked testing method show that compared with the blank control group, the fluorescence intensity of the experiment group gradually increases over testing time, demonstrating that the purified LbuCas13a protein has the expected protein activity.

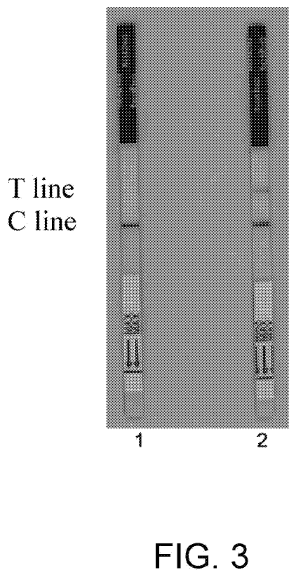

In addition, a Tiosbio Cas12/13 nucleic acid test strip is used for testing, with interpretation criteria as follows: a positive result is achieved if red bands appear at both control (C) and test (T) lines or the C line of the test strip does not show color and the T line shows color; and a negative result is achieved if a red band appears at the C line and the T line does not show color; and an invalid result is achieved if no band appears at the C or T line. As shown in FIG. 3, compared with the negative control group, the experiment group containing the target RNA shows color on the T and C lines, showing a positive result based on the interpretation criteria of the test strip, and demonstrating the expected activity of the LbuCas13a protein.

### Example 4: Preparation and Assembly of Immunochromatographic Test Strip

### 1. Colloidal Carbon-Labeled Antibody

A rabbit anti-biotin antibody was selected as a labeled antibody, and colloidal carbon was used to for conjugate labeling of the antibody. In this experiment, the colloidal carbon-labeled antibody kit from Beijing Nanogold Biotechnology Co., Ltd. was used, for detailed operation steps, a reference can be made to the instruction manual of the kit.

### 2. Preparation of Immunochromatographic Test Strip

The immunochromatographic test strip sequentially included a sample pad, a conjugate pad including a colloidal carbon-labeled rabbit anti-biotin antibody, an NC membrane including T and C lines, and absorbent paper in a sample flowing direction;
when a probe is FAB-polyU-11nt, the T line is formed by a rabbit anti-6-FAM polyclonal antibody, and the C line is formed by a goat anti-rabbit IgG;
when a probe is DB-polyU-11nt, the T line is formed by an anti-digoxigenin antibody, and the C line is formed by a goat anti-rabbit IgG;
when a probe is TB-polyU-11nt, the T line is formed by an anti-TAMRA antibody [5G5], and the C line is formed by a goat anti-rabbit IgG; and
when a probe is CB-polyU-11nt, the T line is formed by an anti-Cy5 antibody [CY5-15], and the C line is formed by a goat anti-rabbit IgG.

For the details of the method for preparing the immunochromatographic test strip, a reference was made to the colloidal carbon immunochromatographic experiment method of Kang Jingru, with slight modifications.

### (1) Sample Pad Treatment

A sample pad material was selected and cut to dimensions of 16 mm×10 cm; the sample pad was placed in a clean tank; about 20 mL of a pre-prepared sample pad treating solution was evenly added to the sample pad such that the sample pad was fully saturated; after soaking for about 10 min, the pad was flipped once. Then, the sample pad was placed in a drying oven for drying 3 h at a temperature set to 37° C., and was temporarily stored in the drying oven for subsequent use in the test strip assembly.

### (2) Scribing on Test Strip

A substrate and an NC membrane were cut to a length of 10 cm; absorbent paper was cut to 2.2 cm×10 cm; a protective film was removed from the substrate, and the NC membrane and the absorbent paper were affixed to the substrate, which was then placed on a slicer; a corresponding antibody or secondary antibody was diluted to an appropriate concentration using a coating buffer (prepared by weighing and adding 0.032 g of Na₂CO₃, 0.059 g of NaHCO₃, 0.878 g of NaCl, 5 g of trehalose and 0.05 g of NaN₃ to 80 mL of ddH₂O, fixing the volume to 100 mL, and then adding 10 mL of methanol); scribing was performed on the NC membrane; and the scribed test strip was dried at 37° C. for 3 h, and temporarily stored in the drying oven for subsequent use in test strip assembly.

### (3) Carbon Spraying on Conjugate Pad

A conjugate pad material was selected and cut to dimensions of 8 mm×10 cm; the prepared colloidal carbon-coated antibody was evenly sprayed to the conjugate pad using a pipette tip. Then, the conjugate pas was dried at 37° C. for 3 h, and stored in a thermostatic drying oven.

### (4) Assembly and Chromatography of Test Strip

After the test strip scribed with the T and C lines was dried, the test strip was assembled with the conjugate pad (8 mm×10 cm), on which the colloidal carbon-labeled antibody was sprayed, and the treated sample pad (16 mm×10 cm), and the assembled test strip was then cut into a piece of 3.4 mm×6 cm using a slicer. Afterwards. The test strip was stored in a sealed aluminum foil bag containing a desiccant.

### 3. Principle of the Present Invention

The present invention is based on a line-free testing method, where the absence of a band indicates a positive result. The nucleic acid testing method of the present invention is based on the cleavage capability of the Cas13a protein, which can be characterized by free RNA. Therefore, an appropriate probe may serve as a primary means of test strip testing. When a testing target is absent (i.e., the probe remains uncleaved), the probe is intact and may be captured at the T line, leading to colloidal carbon accumulation here to show a band. This indicates that the target is not detected, and the result is determined as negative for the target. When a testing target is present, the probe is cleaved, and its colloidal carbon-labeled end migrates freely to the C line, without colloidal carbon accumulation at the T line, indicating a positive result.

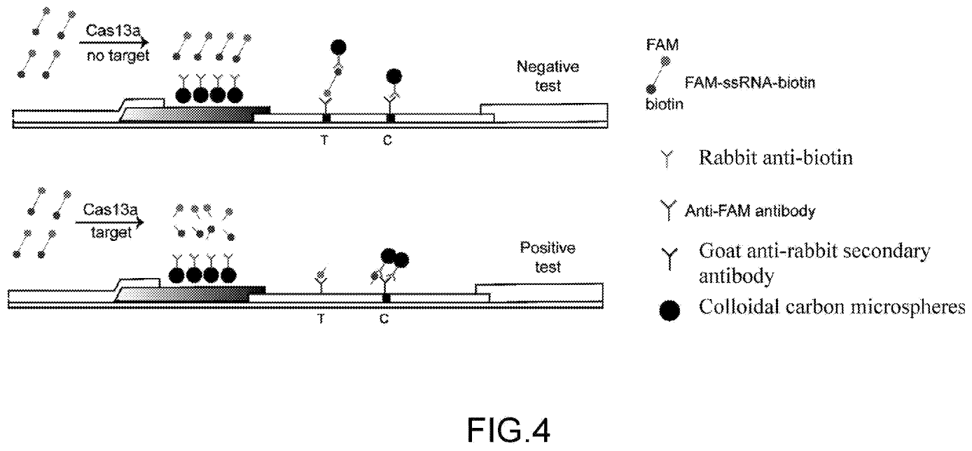

Taking this example and FIG. 4 as an example, the labeled probe is labeled with FAM at one end and biotin at the other end, respectively, a liquid sample to be tested serves as a mobile phase, and the chromatography starts from the sample pad.

Upon reaching the conjugate pad, the biotin at one end of the probe conjugates with the colloidal carbon-labeled rabbit anti-biotin antibody; and when a testing target is present, the probe is cleaved and broken based on the Cas13a-based testing principle, and the colloidal carbon-labeled rabbit anti-biotin antibody migrates freely to the C line and captured by the goat anti-rabbit secondary antibody, forming a black line visible to naked eyes. The anti-FAM antibody at the T line captures the other end of the broken probe labeled with FAM rather than colloidal carbon, and thus, there is no band.

When a testing target is absent, the colloidal carbon-labeled intact probe (with one end labeled with FAM) is captured by the anti-FAM antibody at the T line, thereby forming a black line visible to the naked eyes at the T line.

Regardless of whether the probe in the sample is cleaved or not, the excess colloidal carbon-labeled rabbit anti-biotin antibody continue undergoing chromatography along with the sample, and the goat anti-rabbit secondary antibody at the C line unconditionally binds to the colloidal carbon-labeled rabbit anti-biotin antibody, forming a black line visible to the naked eyes.

### Example 5: Accuracy Verification for LbuCas13a-Based Testing

(1) First, based on the Cas13a-based testing system in Example 3, the concentration of RNase Inhibitor in the system and the concentration of RNA fluorescence reporter analysis were optimized, respectively, and the reaction time for testing was optimized at the same time; and in the optimized reaction system, the final concentration of RNase Inhibitor is 1 U/μL, and the final concentration of RNA fluorescence reporter molecules is 4 nM, with the reaction time of ≥45 min. The optimized LbuCas13a-based testing system is shown in Tables 11 and 12.
(2) The Cas13a protein reaction system was formulated, where two negative control groups and one blank control group were set; in the first negative control group, an equal volume of RNase-Free H₂O was used in place of LbucrRNA and S-gene RNA and added to the reaction system; in the second negative control group, an equal volume of RNase-Free H₂O was used in place of ssRNA and added to the reaction system; and in the blank control group, an equal volume of RNase-Free H₂O was used in place of LwcrRNA, S-gene RNA and LwaCas13a and added to the reaction system. After the preparation of all reaction systems is completed, these reaction systems were added to a clean RNase-free PCR tube to react at 37° C. for 90 min. The prepared test strip was inserted, and the testing result was interpreted within 5-10 min.

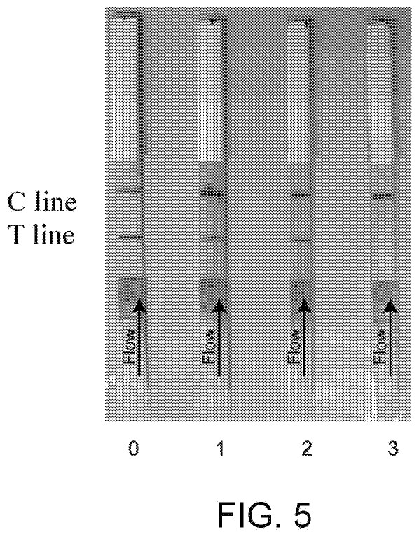

The testing results are shown in FIG. 5, where a positive testing result is determined only if no band exists at the T line when the LbuCas13a protein, LbucrRNA and S-gene RNA coexist, and a negative result is determined if a band appears at the T line when any one of the components is absent. These results further demonstrate the specific cleavage activity of the LbuCas13a protein.
(3) Testing of inactivated SARS-CoV-2 coronavirus samples

In order to ensure the accuracy of the Cas13a-based immunochromatographic testing results, the inactivated SARS-CoV-2 coronavirus nucleic acid samples were tested using Daan Gene 2019-nCOV nucleic test kit (using a fluorescent PCR method) for SARS-CoV-2 coronaviruses and Shanghai Brotek antigen test skit (using a colloidal gold method) for SARS-CoV-2 coronaviruses (2019-nCOV); positive samples were screened. For RNA samples, an LbuCas13a-based immunochromatographic protein testing system was then formulated according to Tables 11 and 12.

TABLE 11
Optimized first LbuCas13a-based testing system

| Component | Volume | |
|---|---|---|
| 10 × Cas13a buffer | 2 | μL |
| Cas13a protein (3 mg/μL) | 2 | μL |
| LbucrRNA1/LbucrRNA2/LbucrRNA3/LbucrRNA4 Mixture | 4 | μL |
| (500 ng/μL) | | |
| ddH₂O | 12 | μL |

After the above reaction systems were prepared, the reaction systems were added to a clean RNase-free PCR tube, and then placed in a PCR instrument to react at 37° C. for 10 min to obtain a step 1 system. After completion of the reaction, a reaction system was added following the table below to perform step 2.

TABLE 12
Optimized first LbuCas13a-based testing system

| Component | Volume | |
|---|---|---|
| Target RNA | 5 | μL |
| 10 × Cas13a buffer | 8 | μL |
| 40 nM RNA reporter | 10 | μL |
| Cas13a/crRNA mixture (from step 1) | 20 | μL |
| RNase Inhibitor (40 U/μL) | 2.5 | μL |
| ddH₂O | 54.5 | μL |

The probe used in this system was TB-polyU-1Int (see Table 6). After the above reaction system was formulated, it was placed in a thermostatic incubator to react at 37° C. for 90 min; and the prepared single-target colloidal carbon test strip was inserted; and a testing result was interpreted within 5-10 min.

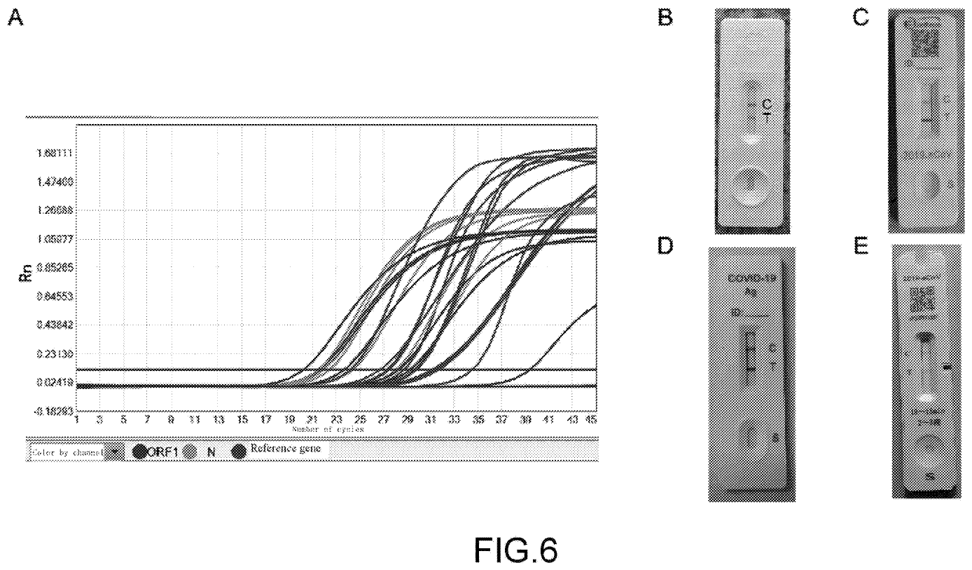

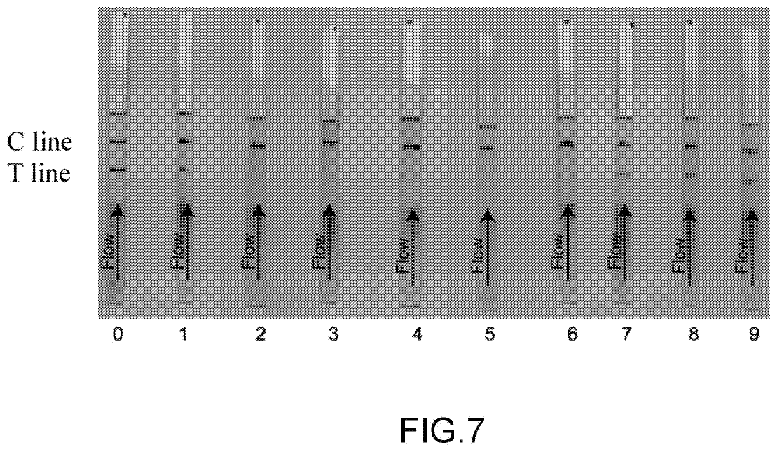

Conclusion: (1) qPCR testing is conducted on the inactivated SARS-CoV-2 coronavirus samples using the SARS-CoV-2 coronavirus test kit (by qPCR) and the SARS-CoV-2 coronavirus kit (by a test strip); a total of three sets of genes are provided for the kits for amplification (as shown in FIG. 6); red indicates human reference genes, green indicates coronavirus N-genes and blue indicates ORF1 genes; the qPCR testing results show that the Ct values of the reference genes are all less than 3, demonstrating valid samples; and both the N and ORF1 genes exhibit amplification curves, each with a Ct value of <35, resulting in the interpretation of a positive testing result. The testing method using the test strip shows bands at both T and C lines, with the interpretation of a positive result, otherwise, a negative result. The analysis of the results reveals a total of 5 positive and 4 negative nucleic testing results.
(2) For the above testing results, the testing results (shown in FIG. 7) of the Cas13a-based immunochromatographic testing method of the present invention are analyzed for the test samples. These testing results show 100% consistency with both the fluorescent PCR method and the test strip method and the accuracy in detecting positive samples, demonstrating the accuracy and specificity of the Cas13a-based immunochromatographic testing method.

### Example 6: Sensitivity Assay of LbuCas13a-based Immunochromatographic Testing

(1) qPCR Quantification Experiment

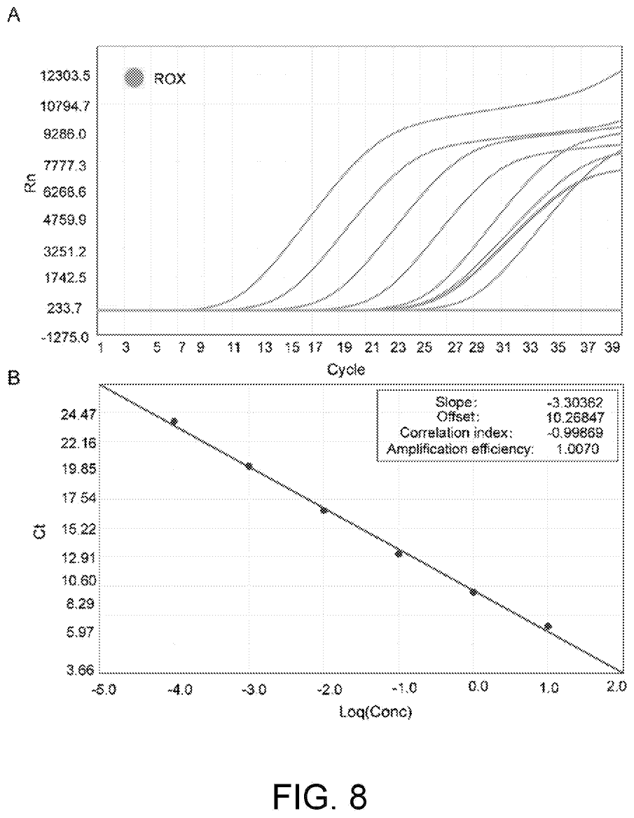

A qPCR quantification experiment was conducted by taking in vitro transcribed RNA as a qPCR standard and SARS-CoV-2 coronavirus-positive sample RNA as a test target, with the testing results shown in FIG. 8, where the standard curves were successfully generated, the test samples fell within the standard curve range, the established standard curve correlation coefficient was 0.99869 (demonstrating high reliability), and the SARS-CoV-2 coronavirus-positive sample RNA showed the average Ct of 21.32.

### (2) Analysis of Droplet Digital PCR Results

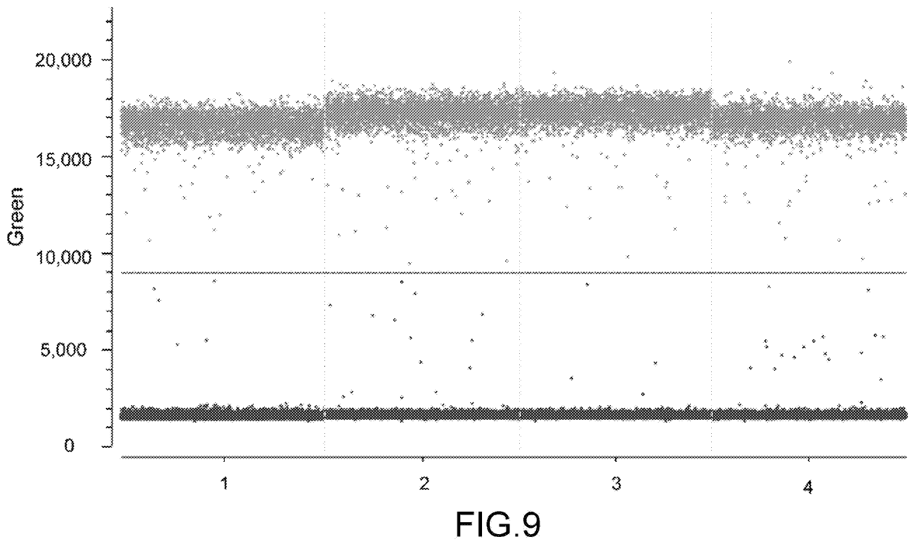

To determine the copy number for the qPCR standard, a digital PCR experiment was conducted. Its one-dimensional testing results in a ROX channel are shown in FIG. 9, where a center line represented a threshold line, with positive droplet distribution above the threshold line and negative droplet distribution below the threshold line; and the longitudinal axis denotes fluorescence intensity, while the horizontal axis denotes the number of a sample well. The analysis of the testing results reveals the presence of a target sequence, with relatively uniform positive droplet distribution, high peak value, consistent results across four replicates, and minimal error. Based on the number of positive samples, the copy number of the qPCR standard is determined as 1.219×10⁸ copies/μL by means of software analysis and computation.

### (3) Sensitivity of LbuCas13a-Based Immunochromatographic Testing

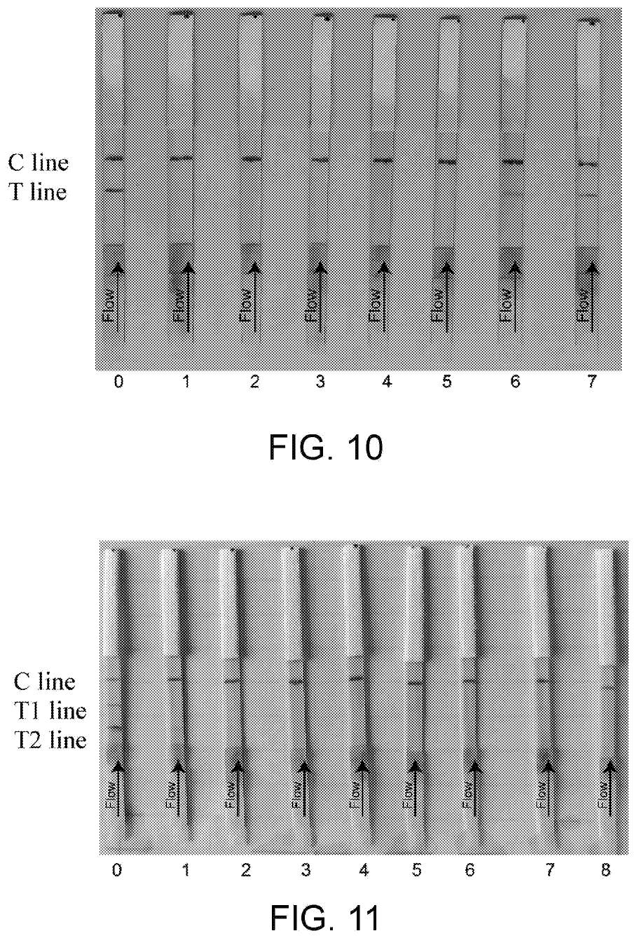

Based on the above determined copy number of the qPCR standard, the copy number of the test samples may be estimated as 6108 copies/μL by conversion of the Ct values of the qPCR standard curves. This sample was subjected to gradient dilution, and was tested using the Cas13a-based immunochromatographic testing method to determine the sensitivity, with the results shown in FIG. 10.

The analysis of the results reveals that when the copy number of sample concentration decreases to 190 copies/μL, colloidal carbon accumulation occurs at the test line during the Cas13a-based immunochromatographic testing, leading to the interpretation of a negative result. Therefore, the minimum detection limit of the Cas13a-based immunochromatographic testing method is inferred as 381.75 copies/μL.

### Example 7: Testing of Inactivated SARS-CoV-2 Coronavirus Samples

For the SARS-CoV-2 coronavirus variant EG.5.1.1, crRNA was designed targeting the N-gene, where the crRNA was prepared by referring to the in vitro transcription described in Example 1, and primers were listed in Table 13 and synthesized by Anhui General Biology Co., Ltd. The detailed steps for preparation of double-stranded products and in vitro transcription were the same as those in Example 2.

TABLE 13
crRNAs of SARS-CoV-2 coronavirus samples

| Primer | Sequence (5′-3′) |
|---|---|
| N1-CR | GACCACCCCAAAAATGAAGGGGACTAAAACTGTCCTTTTTA |
| | GGCTCTGTTGGTGGGA |
| N1-FOR | AATTAACCCTCACTAAAGGGGACCACCCCAAAAATGAAGGG |
| N1-REV | TCCCACCAACAGAGCCTAAAA |
| N2-CR | GACCACCCCAAAAATGAAGGGGACTAAAACTGAACTGTTGC |
| | GACTACGTGATGAGGA |
| N2-FOR | AATTAACCCTCACTAAAGGGGACCACCCCAAAAATGAAGGG |
| N2-REV | TCCTCATCACGTAGTCGCAAC1 |

The prepared crRNA of the SARS-CoV-2 coronavirus and the LbuCas13a protein were used to formulate the reaction system by referring to Tables 14 and 15.

TABLE 14
First Cas13a-based immunochromatographic testing
system for SARS-CoV-2 coronaviruses

| Component | Volume | |
|---|---|---|
| 10 × Cas13a buffer | 2 | μL |
| Cas13a protein (3 mg/μL) | 2 | μL |
| N1crRNA/N2crRNA Mixture (500 ng/μL) | 2 | μL |
| ddH₂O | 14 | μL |

After the above reaction systems were prepared, the reaction systems were added to a clean RNase-free PCR tube, and then placed in a PCR instrument to react at 37° C. for 10 min to obtain a step 1 system.

TABLE 15
Second immunochromatographic testing
system for SARS-CoV-2 coronaviruses

| Component | Volume | |
|---|---|---|
| SARS-CoV-2 coronavirus RNA | 5 | μL |
| 10 × Cas13a buffer | 8 | μL |
| 40 nM RNA reporter | 10 | μL |
| Cas13a/crRNA mixture (from step 1) | 20 | μL |
| RNase Inhibitor (40 U/μL) | 2.5 | μL |
| ddH₂O | 54.5 | μL |

The probe used in this system was TB-polyU-11nt (see Table 6), and after formulation, the reaction system was placed in an PCR instrument to react at 37° C. for 90 min and then at 4° C. for 2 min, followed by terminating the reaction.

After completion of the reaction for sample testing, a dual-target immunochromatographic test strip was inserted in 50 μL of reaction system in each case, and the test result was interpreted within 5-10 min.

Eight inactivated SARS-CoV-2 coronavirus-positive RNA samples and 8 inactivated influenza A H₃N2-positive RNA samples were tested using dual-target Cas13a-based immunochromatographic test strips, with the testing results shown in FIG. 11. A multi-target immunochromatography test strip was constructed by selecting and laying a corresponding capture antibody based on the probe of the testing system, where one probe corresponded to one test target, and a plurality of T lines might be laid for the prepared colloidal carbon test strip. In this way, one probe corresponded to one test target, and different probes were captured by different antibodies, such that a plurality of testing systems was formulated for the experiment, the samples were added respectively for reaction, and after completion of the reaction, all the systems were mixed thoroughly and tested on the same test strip to present testing results, thereby achieving multi-target testing.

The results show no positive detection of influenza samples on the test strip 1, and no positive detection of SARS-CoV-2 coronavirus on the test strip 7, suggesting potentially low sample concentrations. All the positive samples having other numbers are successfully detected. This validates the specificity and feasibility of the multi-target Cas13a-based immunochromatographic testing.

What is claimed is:

1. A probe set for nucleic acid testing of SARS-CoV-2 coronaviruses, wherein a crRNA sequence for nucleic acid testing of SARS-CoV-2 coronaviruses is any one of the following: LbucrRNA1, LbucrRNA2, LbucrRNA3 and LbucrRNA4 for an S gene, as well as N1crRNA and N2crRNA for an N gene of a mutant strain EG.5.1.1, with sequences as follows:

| Name | Sequence (5′-3′) |
|---|---|
| LbucrRNA1 | GACCACCCCAAAAATGAAGGGGACTAAAACGAATTCC |
| | AAGCTATAACGCAGCCTGTAA (SEQ ID NO: 1) |
| LbucrRNA2 | GACCACCCCAAAAATGAAGGGGACTAAAACCTTGCTG |
| | TGGAAGAAAGTGAGTCTTGA (SEQ ID NO: 2) |
| LbucrRNA3 | GACCACCCCAAAAATGAAGGGGACTAAAACGCGATTT |
| | GTCTGACTTCATCACCTCTA (SEQ ID NO: 3) |
| LbucrRNA4 | GACCACCCCAAAAATGAAGGGGACTAAAACCCTATCA |
| | ATTTGCACTTCAGCCTCAAC (SEQ ID NO: 4) |
| N1crRNA | GACCACCCCAAAAATGAAGGGGACTAAAACTGTCCTT |
| | TTTAGGCTCTGTTGGTGGGA (SEQ ID NO: 5) |
| N2crRNA | GACCACCCCAAAAATGAAGGGGACTAAAACTGAACTG |
| | TTGCGACTACGTGATGAGGA (SEQ ID NO: 6) |

2. A kit for nucleic acid testing of SARS-CoV-2 coronaviruses, comprising the probe set for nucleic acid testing of SARS-CoV-2 coronaviruses according to claim 1.

3. A kit for nucleic acid testing of SARS-CoV-2 coronaviruses, comprising a line-free colloidal carbon immunochromatographic test strip and a CRISPR reaction system, wherein the CRISPR reaction system comprises the probe set for nucleic acid testing of SARS-CoV-2 coronaviruses according to claim 1; and the line-free colloidal carbon immunochromatographic test strip sequentially comprises a sample pad, a conjugate pad comprising a colloidal carbon-labeled rabbit anti-biotin antibody, an NC membrane comprising T and C lines, and absorbent paper in a sample flowing direction.

4. The kit for nucleic acid testing of SARS-CoV-2 coronaviruses according to claim 3, wherein
when a probe is FAB-polyU-11nt, the T line is formed by a rabbit anti-6-FAM polyclonal antibody, and the C line is formed by a goat anti-rabbit IgG;
when a probe is DB-polyU-11nt, the T line is formed by an anti-digoxigenin antibody, and the C line is formed by a goat anti-rabbit IgG;
when a probe is TB-polyU-11nt, the T line is formed by an anti-TAMRA antibody [5G5], and the C line is formed by a goat anti-rabbit IgG;
when a probe is CB-polyU-11nt, the T line is formed by an anti-Cy5 antibody [CY5-15], and the C line is formed by a goat anti-rabbit IgG; and
each probe has a sequence as follows:

| Name | Sequence (5′-3′) |
|---|---|
| FAB-polyU-11 nt | FAM-UUUUUUUUUUU-Biotin |
| TB-polyU-11 nt | TAMRA- |
| | UUUUUUUUUUU-Biotin |
| DB-polyU-11 nt | Dig-UUUUUUUUUUU-Biotin |
| CB-polyU-11 nt | Cy5-UUUUUUUUUUU-Biotin |

5. The kit for nucleic acid testing of SARS-CoV-2 coronaviruses according to claim 3, wherein the CRISPR reaction system is as follows,
First system for S gene

| Component | Volume | |
|---|---|---|
| 10 × Cas13a buffer | 2 | μL |
| Cas13a protein (3 mg/μL) | 2 | μL |
| LbucrRNA1/LbucrRNA2/LbucrRNA3/LbucrRNA4 Mixture | 4 | μL |
| (500 ng/μL) | | |
| ddH₂O | 12 | μL |

First system for N gene

| Component | Volume | |
|---|---|---|
| 10 × Cas13a buffer | 2 | μL |
| Cas13a protein (3 mg/μL) | 2 | μL |
| N1crRNA/N2RNA Mixture (500 ng/μL) | 2 | μL |
| ddH₂O | 14 | μL |

after being prepared, the first system is added to a clean RNase-free PCR tube, and then placed in a PCR instrument to react at 37° C. for 10 min to obtain a step 1 system, and after completion of reaction, a Cas13a/crRNA mixture is obtained and added to a second system for step 2,
Second system for S or N gene

| Component | Volume | |
|---|---|---|
| Target RNA | 5 | μL |
| 10 × Cas13a buffer | 8 | μL |
| 40 nM RNA reporter | 10 | μL |
| Cas13a/crRNA mixture (from step 1) | 20 | μL |
| RNase Inhibitor (40 U/μL) | 2.5 | μL |
| ddH₂O | 54.5 | μL |

after being prepared, the second system is placed in the PCR instrument to react at 37° C. for 90 min and then at 4° C. for 2 min to terminate the reaction, the prepared colloidal carbon immunochromatographic test strip is inserted, and test results are read within 5-10 min.

6. Use of the kit according to claim 2 in nucleic acid testing of SARS-CoV-2 coronaviruses.

7. Use of the kit according to claim 3 in nucleic acid testing of SARS-CoV-2 coronaviruses.

8. Use of the kit according to claim 4 in nucleic acid testing of SARS-CoV-2 coronaviruses.

9. Use of the kit according to claim 5 in nucleic acid testing of SARS-CoV-2 coronaviruses.


SEQUENCE LISTING (6 sequences)

SEQ ID NO: 1 - RNA, 58 nt, synthetic construct
  source 1..58: mol_type = other RNA
gaccaccccaaaaatgaaggggactaaaacgaattccaagctataacgcagcctgtaa

SEQ ID NO: 2 - RNA, 57 nt, synthetic construct
  source 1..57: mol_type = other RNA
gaccaccccaaaaatgaaggggactaaaaccttgctgtggaagaaagtgagtcttga

SEQ ID NO: 3 - RNA, 57 nt, synthetic construct
  source 1..57: mol_type = other RNA
gaccaccccaaaaatgaaggggactaaaacgcgatttgtctgacttcatcacctcta

SEQ ID NO: 4 - RNA, 57 nt, synthetic construct
  source 1..57: mol_type = other RNA
gaccaccccaaaaatgaaggggactaaaaccctatcaatttgcacttcagcctcaac

SEQ ID NO: 5 - RNA, 57 nt, synthetic construct
  source 1..57: mol_type = other RNA
gaccaccccaaaaatgaaggggactaaaactgtcctttttaggctctgttggtggga

SEQ ID NO: 6 - RNA, 57 nt, synthetic construct
  source 1..57: mol_type = other RNA
gaccaccccaaaaatgaaggggactaaaactgaactgttgcgactacgtgatgagga